(19) United States
(12) Patent Application Publication
(10) Pub. No.: US 2021/0241125 A1
(43) Pub. Date: Aug. 5, 2021

(54) APPAREL THERMAL COMFORT PREDICTION SYSTEM

(71) Applicant: 3M INNOVATIVE PROPERTIES COMPANY, St Paul, MN (US)

(72) Inventors: Kristy A. Jost, Woodbury, MN (US); Mark V. Riofski, Eagan, MN (US); Jonathan B. Arthur, Hudson, WI (US); Kenneth A. Cox, Woodbury, MN (US); Karl W. Bloedorn, Oakdale, MN (US)

(21) Appl. No.: 17/053,915

(22) PCT Filed: May 3, 2019
(86) PCT No.: PCT/IB2019/053643

§ 371 Date: Nov. 9, 2020

Related U.S. Application Data
(60) Provisional application No. 62/669,124, filed on May 9, 2018.

(51) Int. Cl. G06N 5/02; G06N 7/00; G06N 20/10; A41D 1/00; A41D 13/005

(52) U.S. Cl. CPC G06N 5/02; G06N 7/005; G16H 40/63; A41D 1/002; A41D 13/005; G06N 20/10

(57) ABSTRACT

A method includes receiving sensor information generated by a sensor embedded in an article of apparel. The method includes determining, based at least in part on the sensor information, a thermal comfort score indicative of a probability that a user of the article of apparel will be comfortable at a future time. The method also includes determining, based on the thermal comfort score, whether the user of the article of apparel is likely to be comfortable at the future time. The method further includes responsive to determining that the user of the article of apparel is not likely to be comfortable at the future time, performing an operation.

### TECHNICAL FIELD

The present disclosure relates generally to articles of apparel and computing systems related to use of articles of apparel.

### BACKGROUND

Articles of apparel are often designed to keep a user of the article warm in cold temperatures or keep the user cool in warm temperatures. In many cases, a user may inadvertently choose to wear an article of apparel that is not designed for the weather or activity of the user. Moreover, the user may not recognize he or she is too hot or too cold until his or her core temperature is no longer in a safe and/or comfortable temperature range.

### SUMMARY

The present disclosure describes techniques for predicting thermal comfort of a person wearing an article of apparel. For example, various implementations of an article of apparel are described that include one or more sensors in communication with a computing device (e.g., a mobile phone, server system, etc.). The computing device is configured to determine whether a user of the particular article of apparel (e.g., an individual wearing the article of apparel) is likely to be at a comfortable or uncomfortable temperature at a future time. The computing device may determine whether the user is likely to be comfortable at the future time based at least in part on information generated by the sensors. For example, the sensors may monitor the user's physiological characteristics (e.g., heart rate, sweat, body temperature, etc.) or environmental characteristics (e.g., air temperature, humidity, etc.). As another example, the computing device may determine whether the user is likely to be comfortable based on user information. For example, the computing device may receive historical user comfort information (e.g., via user input) indicating whether the user was comfortable at some time in the past and corresponding historical sensor information, and may determine whether the user is likely to be comfortable in the future based on the current sensor information, historical sensor information and the corresponding historical comfort information. In some examples, the computing device determines whether the user is likely to be comfortable based on information about the article being worn by the user (e.g., type of material of the article, age of the article, etc.). By predicting whether the user is likely to be comfortable while wearing the article at some time in the future, the computing device may reduce the likelihood that the user's core body temperature reaches an uncomfortable or unsafe level, which may improve the health and/or safety of a user wearing the article of apparel. In some examples, the computing device may predict whether the user is likely to be comfortable based on historical user information associated with that user, which may enable the computing device to tailor the prediction based on the individual user's tolerances and health, which may provide more accurate predictions for the individual user.

In one example, a method includes receiving, by at least one processor, sensor information generated by a sensor embedded in an article of apparel. The method also includes determining, by the at least one processor, based at least in part on the sensor information, a thermal comfort score indicative of a probability that an individual wearing the article of apparel will be comfortable at a future time. The method further includes determining, by the at least one processor, based on the thermal comfort score, whether the user of the article of apparel is likely to be comfortable at the future time. The method includes responsive to determining that the user of the article of apparel is not likely to be comfortable at the future time, performing an operation.

In another example, a system includes an article of apparel comprising a sensor; at least one processor; and a memory. The memory includes instructions that, when executed by the at least one processor, cause the at least one processor to: receive sensor information generated by the sensor and determine, based at least in part on the sensor information, a thermal comfort score indicative of a probability that a user of the article of apparel will be comfortable at a future time. Execution of the instructions further cause the at least one processor to determine, based on the thermal comfort score, whether the user of the article of apparel is likely to be comfortable at the future time; and responsive to determining that the user of the article of apparel is not likely to be comfortable at the future time, perform an operation.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a block diagram illustrating an example system that is configured to predict whether a user of an article of apparel will be comfortable at a future time, in accordance with various techniques of this disclosure.

FIG. 2 is a block diagram illustrating an example computing device that is configured to predict whether a user of an article of apparel will be comfortable at a future time, in accordance with various techniques of this disclosure.

FIG. 3 is a block diagram illustrating an example computing device that is configured to predict whether a user of an article of apparel will be comfortable at a future time, in accordance with various techniques of this disclosure.

FIG. 4 is a block diagram illustrating an example computing device that is configured to predict whether a user of an article of apparel will be comfortable at a future time, in accordance with various techniques of this disclosure.

FIG. 5 is a flow chart illustrating example operations performed by one or more computing devices that are configured to predict whether a user of an article of apparel will be comfortable at a future time, in accordance with various techniques of this disclosure.

### DETAILED DESCRIPTION

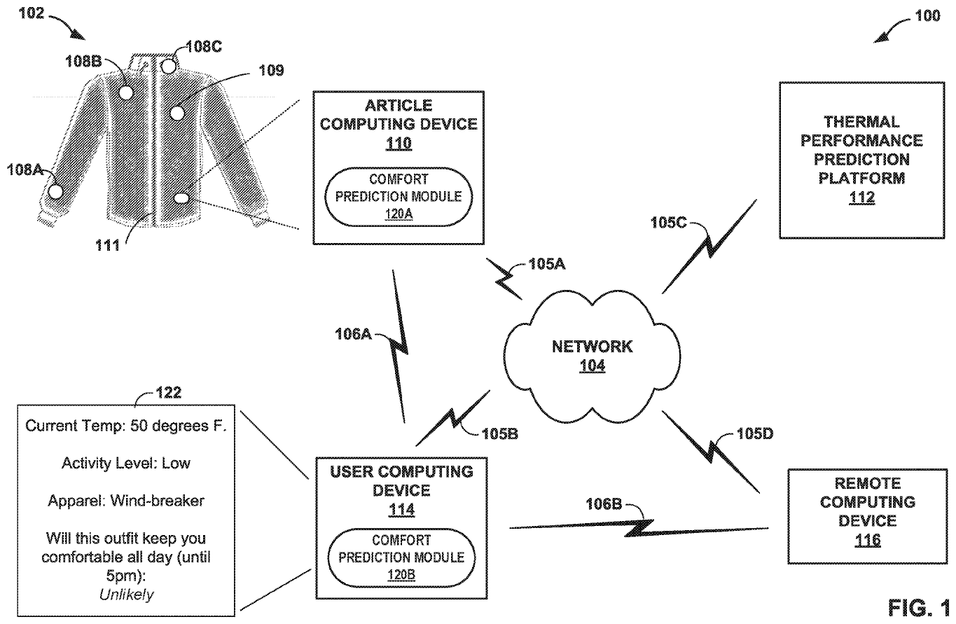

FIG. 1 is a block diagram illustrating an example system that is configured to predict whether a user of an article of apparel will be comfortable at a future time, in accordance with one or more aspects of the present disclosure. System 100 of FIG. 1 includes article 102, which includes an article computing device 110. In this example, system 100 further includes thermal performance prediction platform (TPPP) 112, user computing device 114, and remote computing device 116.

User computing device 114 and remote computing device 116 are examples of a computing device, such as a smartphone, a computerized wearable device (e.g., a watch, eyewear, ring, necklace, etc.), laptop, desktop, or any other type of computing device. In some examples, user computing device 114 and remote computing device 116 are configured to send and receive information (also referred to as data) via a network, such as network 104.

Network 104 represents any public or private communication network, for instance, cellular, WiFi®, LAN, mesh network, and/or other types of networks for transmitting information between computing systems, servers, and computing devices. Network 104 may provide computing devices, such as article computing device 110 of article 102, TPPP 112, and computing devices 114, 116 with access to the Internet, and may allow the computing devices to communicate with each other. Article computing device 110, TPPP 112, and computing devices 114, 116 may each be operatively coupled to network 104 using respective network links, such as links 105A-105D (collectively, “network links 105”). Network links 105 may be any type of network connections, such as wired or wireless connections.

In some examples, one or more computing devices of system 100 may exchange information with another computing device without the information traversing network 104. For example, article computing device 110 may communicate with user computing device 114 using direct link 106A. Similarly, user computing device 114 may communicate with remote computing device 116 via direct link 106B. Direct links 106A, 106B may be any communication protocol or mechanism capable of enabling two computing devices to communicate directly (i.e., without requiring a network switch, hub, or other intermediary network device), such as Bluetooth®, Wi-Fi Direct®, near-field communication, etc.

Article 102 may include any type of apparel, such as a jacket, shirt, trousers or pants, gloves, hat, shoes, etc. that may be worn by a human or animal. Article 102 includes one or more sensors, such as sensors 108A-108C (collectively, sensors 108). Sensors 108 may include one or more movement sensors (e.g., accelerometers, gyroscopes, etc.), temperature sensors (e.g., thermistors), light sensors (e.g., ambient light sensors), humidity sensors (e.g., a hygrometer), position sensors (e.g., GPS), pressure sensors (e.g., air pressure, touch sensors), heart rate sensor, or any other type of sensor. Sensors 108 may generate sensor information indicative of a sensed user physiological characteristic (e.g., heart rate, breathing rate, body temperature, presence or amount of sweat, motion, etc.) and/or environmental characteristic (e.g., air temperature, humidity, location, etc.) and may output the sensor information. Sensors 108 may be located at various locations of article 102. For example, in the example of FIG. 1, article 102 is illustrated as long-sleeved garment (e.g., a jacket, a sweater, sweatshirt, etc.) and sensors 108 may be located near the wrist, torso, and collar. In other examples, sensors 102 may be located at areas of the article that cover the user's mid-section, hand, leg, ankle, foot, etc. In some examples, sensors 108 may be located on an inner surface of article 102, outer surface of article 102 (e.g., front or back), an inner portion of article 102 (e.g., between the inner and outer surfaces), or any combination therein.

Article 102 may include one or more temperature control devices 109. In some examples, temperature control devices 109 include active temperature control devices (e.g., heating or cooling elements) used to directly affect the temperature on the interior of article 102. As another example, temperature control device 109 may include passive temperature control devices, such as aperture control devices 111, that indirectly affect the temperature on the interior of article 102. Aperture control devices include zippers, drawstrings (e.g., a drawstring around a hood, wrist, waist, etc.), or other devices used to control airflow into and out of article 102.

In some examples, article computing device 110 and user computing device 114 each include a respective comfort prediction module 120A, 120B (collectively, “comfort prediction modules 120”). Although not shown, TPPP 112 and/or remote computing device 116 may include similar components or modules as computing devices 110, 114. Modules 120 may perform operations described using hardware, hardware and firmware, hardware and software, or a mixture of hardware, software, and firmware residing in and/or executing at computing devices 110, 114. Computing devices 110, 114 may execute modules 120 with one or multiple processors or multiple devices. Computing devices 110, 114 may execute modules 120 as virtual machines executing on underlying hardware. Modules 120 may execute as one or more services of an operating system or computing platform. Modules 120 may execute as one or more executable programs at an application layer of a computing platform.

Comfort prediction modules 120 may determine whether a user is likely to be comfortable or uncomfortable in the future while wearing article 102. In some examples, comfort prediction modules 120 determine whether the user is likely to be comfortable at a particular future time (e.g., 2 hours from the current time, 3 pm, etc.) while wearing article 102. As another example, comfort prediction modules 120 determine a future time at which the user is not likely to be comfortable while wearing article 102. For example, comfort prediction module 120 may determine that the user is likely to transition from comfortable to uncomfortable at a future time (e.g., within approximately 30 minutes of the current time) while wearing article 102. Comfort prediction modules 120 determine whether a user of article 102 (e.g., an individual wearing article 102) is likely to be comfortable at a future time while wearing article 102 by determining a thermal comfort score and comparing the thermal comfort score to a threshold comfort score.

In some examples, comfort prediction modules 120 determine a thermal comfort score that is indicative of a probability that a user of article 102 will be comfortable (e.g., the user is not too hot or too cold) at a future time. Comfort prediction modules 120 may determine the thermal comfort score based on user physiological characteristics (e.g., heart rate, breathing rate, body temperature, etc.) of the user that is wearing article 102, environmental characteristics (e.g., air temperature, humidity, ambient light, etc.), properties of the article worn by the user (e.g., type of material of the article, age of the article, etc.), user information (e.g., historical comfort information, user activity information, etc.), or any combination therein.

For example, comfort prediction modules 120 may determine the thermal comfort score based at least in part on sensor information received from one or more sensors 108 installed within wearing article 102. In some examples, sensors 108 generate sensor information indicative of the user's physiological characteristics and/or environmental characteristics. For example, sensor 108A may include a temperature sensor that detects the user's body temp, the temperature external to article 102 (e.g., the ambient air temperature), the temperature internal to article 102 (e.g., the air temp between a wearer's body and the inside surface of article 102), or a combination therein. Comfort prediction module 120A may receive the temperature information from sensor 108A and may determine the thermal comfort score based on the received temperature information. For example, comfort prediction module 120A may assign a relatively high thermal comfort score (e.g., 90 out of 100, which may indicate the user of article 102 is likely comfortable) when the temperature is a first temperature and may assign a different (e.g., lower) thermal comfort score as the temperature increases (e.g., which may indicate the user is likely uncomfortably hot) or as the temperature decreases (e.g., which may indicate the user is likely uncomfortably cold).

In some examples, article computing device 110 may send the sensor information generated by one or more of sensors 108 to another computing device, such as computing user computing device 114, for processing. User computing device 114 may, for example, receive the sensor information and may determine the thermal comfort score. For example, comfort prediction module 120B of user computing device 114 determines the thermal comfort score in a similar manner as described for comfort prediction module 120A of article computing device 110.

In some examples, comfort prediction modules 120 determine or assign a thermal comfort score based on one or more sensors disposed on an inner (also referred to as interior) surface of article 102, one or more sensors disposed on an outer (also referred to as exterior) surface of article 102, one or more sensors disposed between an inner surface and an outer surface of article 102, or a combination therein. For example, comfort prediction modules 120 may receive ambient light information from an ambient light sensor disposed on an exterior surface of article 102 and heat flux information from a heat flux sensor disposed between an inner surface and outer surface of article 102. For instance, a user of article 102 may feel more comfortable when the ambient light sensor detects more light (e.g., when in the exposed sunlight), such that comfort prediction modules 120 may assign a higher thermal comfort score when the ambient light information from the ambient light sensor indicates higher levels of light. In some instances, a user of article 102 may feel more comfortable when a heat flux sensor detects a relatively low amount of heat transferred between an inner surface and outer surface of article 102 (e.g., which may indicate the user of article 102 is not losing or gaining heat). For instance, comfort prediction modules 120 may assign a first thermal comfort score when the heat flux information from the heat flux sensor indicates a relatively small amount of heat dissipation through article 102 and a second (e.g., lower) thermal comfort score when the heat flux information indicates a different (e.g., higher) amount of heat dissipation. In some examples, comfort prediction modules 120 receive sensor information generated by sensors of another computing device (e.g., additionally or alternatively to sensor information generated by sensors 108 of article 102), such as sensors of user computing 114 (e.g., an accelerometer) and may assign the thermal comfort score based on such sensor information.

In some examples, comfort prediction module 120 may determine the thermal comfort score based at least in part on information stored in a data structure, such as historical sensor information, historical user information, article information, or a combination therein. For example, comfort prediction modules 120 may receive historical information by querying a data structure or data store. Comfort prediction modules 120 may assign the thermal comfort score based in part on a comparison of the historical information to current sensor information. For example, comfort prediction modules 120 may query the data structure to identify instances where the historical sensor information is similar to the current sensor information, and determine, based on historical user comfort information for those instances, whether the user was comfortable in similar previous instances. Similarly, in some examples, comfort prediction modules 120 receive article information (e.g., by querying a data structure) indicative of the article's properties (e.g., age, type of material, etc.) and assign the thermal comfort score based the article information. In some examples, comfort prediction modules 120 determine the thermal comfort score based on information from other computing devices, such as forecasted weather information (e.g., received from TPPP 112). For example, comfort prediction modules 120 may query a weather provider for weather information and assign the thermal comfort score based on the current temperature, forecasted temperature for a later time, or both.

Comfort prediction modules 120 may determine whether the thermal comfort score satisfies (e.g., is greater than or equal to) a threshold comfort score. Comfort prediction module 120 may determine the threshold comfort score by querying a memory device (e.g., the threshold comfort score may be hard-coded). In some examples, comfort prediction modules 120 dynamically determine the threshold comfort score. For example, comfort prediction modules 120 may determine the threshold comfort score based on information received from one or more sensors 108. For instance, a user may be relatively more prone to feeling cold when the user is not physically active, but may be less susceptible to feeling cold when physically active. Comfort prediction modules 120 may assign a higher threshold comfort score in response to determining that the movement information indicates the user is relatively inactive (e.g., is not engaged in a physical activity) or a lower threshold comfort score in response to determining that the movement information indicates the user is physically active.

In some examples, comfort prediction modules 120 determine or predict that the user is likely to be comfortable while wearing article 102 at a future time in response to determining that the thermal comfort score satisfies (e.g., is greater than or equal) the threshold comfort score. Similarly, comfort prediction modules 120 may determine or predict that the user is not likely to be comfortable at the future time in response to determining that the thermal comfort score does not satisfy (e.g., is less than) the threshold comfort score.

Responsive to determining or predicting that the user is not likely to be comfortable while wearing article 102 at a future time, comfort prediction modules 120 causes a computing device to perform one or more operations. In some examples, the one or more operations include outputting a notification indicative of the prediction that the individual is not likely to be comfortable at some time in the future. As one example, comfort prediction modules 120 may output the notification to an output device of article 102 (e.g., a graphical, audio, and/or haptic user interface device). For example, comfort prediction module 120A of article computing device 110 may output the notification to an audio device of article 102, such that the audio device may output an audible alert indicating the user is not likely to be comfortable at a future time. As another example, comfort prediction module 120B of user computing device 114 may output a notification for display by user computing device 114. For example, user computing device 114 may output a graphical user interface (GUI), such as GUI 122, that includes the notification. In the example illustrated in FIG. 1, user computing device 114 outputs a GUI 122 that includes an alert indicating whether a user of article 102 is likely to be comfortable. For example, the alert may include a message indicating that the user of article 102 is unlikely to be uncomfortable at a predetermined time in the future (e.g., 5 pm). As another example, the alert may include a message indicating a particular time at which the user of article 102 is predicted to be uncomfortable (e.g., an approximate time by which the individual is likely to transition from comfortable to uncomfortable). In some examples, the alert includes additional information, such as information indicating the user's current activity level, the type of article 102, a temperature (e.g., external air temperature and/or internal temperature).

In another example, comfort prediction modules 120 may output a notification indicating that the individual not likely to be comfortable to another computing device. For example, comfort prediction module 120A of article computing device 110 may output a notification to user computing device 114. User computing device 114 may receive the notification and output a graphical user interface (GUI) indicative of the notification. For example, as illustrated in FIG. 1, user computing device 114 may output GUI 122. Similarly, comfort prediction module 120B of user computing device 114 may output a notification to remote computing device 116, such that an output device of remote computing device 116 may output an alert (e.g., graphical, audible, haptic) to a user of remote computing device 116. In this way, a user of remote computing device 116 (e.g., a worker's supervisor) may receive a notification that the user (e.g., a worker) of article 102 is likely to be uncomfortable and take action to improve worker comfort and safety.

In addition, article computing device 110 may perform one or more operations in response to a prediction that the individual is not likely to be uncomfortable, such as adjusting operation of article 102. In some example, article computing device 110 adjusts (e.g., automatically) at least one temperature control device 109 of the article 102. For example, article computing device 110 may automatically activate a temperature control device (e.g., a heating or cooling device). For example, article computing device 110 may turn-on, turn-off, or otherwise adjust the temperature of a heating or cooling device of article 102. As another example, article computing device 110 may automatically output a command to adjust an aperture control device 111, such as a zipper or drawstring. For example, article computing device 110 may output a command to actuate (e.g., open or close) a zipper or adjust (e.g., tighten) a drawstring. In some examples, aperture control device 111 may receive the command and regulate (e.g., open, close, tighten, loosen, etc.) aperture control device 111 in response to receiving the command.

In some examples, article computing device 110 and/or user computing device 114 may send information to TPPP 112, receive information from TPPP 112, or both. For example, article computing device 110, user computing device 114, or both may send sensor information to TPPP 112. TPPP 112 may store sensor information for a plurality of users and articles 102. In some examples, TPPP 112 may store user information (e.g., historical user information indicating whether the user is uncomfortable at various times). TPPP 112 may send information, such as an insulation rating (e.g., R value) corresponding to article 102, weather information (e.g., current and/or predicted temperature information), or any other information to comfort prediction modules 120 that may be used to generate a thermal comfort score. In some examples, TPPP 112 determines the thermal comfort score, determines whether the thermal comfort score satisfies a threshold comfort score, and outputs notifications (e.g., to article computing device 110, user computing device 114, and/or remote computing device 116) in response to determining that the thermal comfort score does not satisfy the threshold comfort score.

In this way, techniques of disclosure enable a computing device to predict whether a user of a particular article of clothing is likely to be comfortable or uncomfortable at some point in the future. By automatically performing an operation (e.g., outputting a notification and/or adjusting at least one temperature control device) in response to determining the individual is likely to be uncomfortable, the computing device may reduce the likelihood that the individual's core body temperature reaches an uncomfortable or unsafe level, which may improve the health and/or safety of a user of the article of apparel.

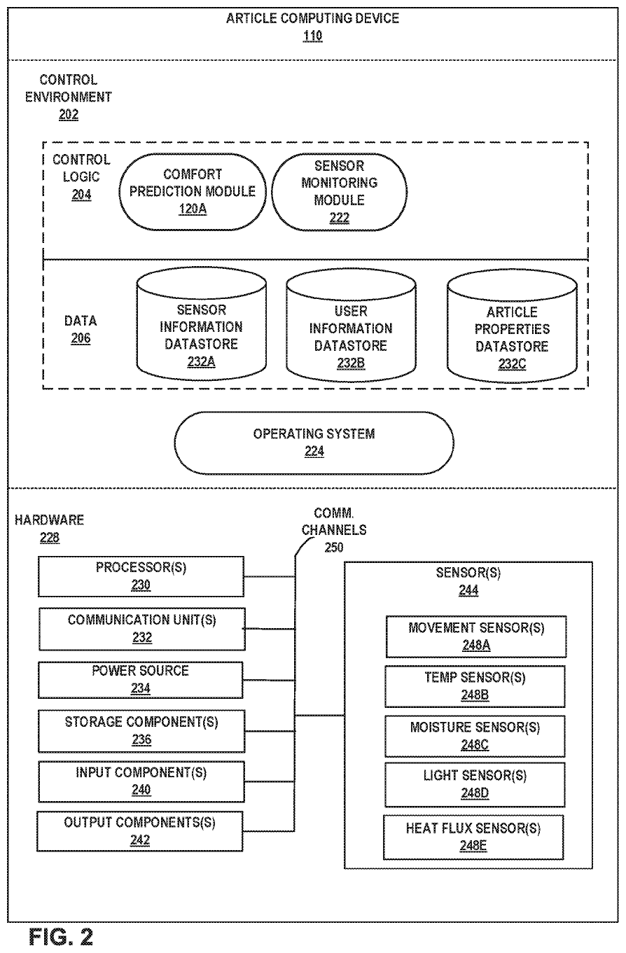

FIG. 2 is a block diagram illustrating an example article computing device that is configured to predict whether a user of an article of apparel will be comfortable at a future time, in accordance with various techniques of this disclosure. FIG. 2 illustrates only one particular example of article computing device 110. Many other examples of article computing device 110 may be used in other instances and may include a subset of the components illustrated in FIG. 2 and/or may include additional components not shown in FIG. 2.

Article computing device 110 may be logically divided into control environment 202 and hardware 228. Hardware 228 may include one or more hardware components that provide an operating environment for components executing in control environment 202. Control environment 202 may include operating system 224, which or may not operate with higher privileges than other components executing in control environment 202.

As shown in FIG. 2, hardware 228 includes one or more processors 230, communication units 232, power source 234, storage components 236, input components 240, output components 242, and sensors 244. Processors 230, communication units 232, power source 234, storage components 236, input components 232, output components 242, and sensors 244 may each be interconnected by one or more communication channels 250. Communication channels 250 may interconnect each of the components 230, 232, 234, 236, 240, 242, and 244 for inter-component communications (physically, communicatively, and/or operatively). In some examples, communication channels 250 may include a hardware bus, a network connection, one or more inter-process communication data structures, or any other components for communicating data between hardware and/or software.

One or more processors 230 may implement functionality and/or execute instructions within article computing device 110. For example, processors 230 may receive and execute instructions stored by storage components 236 that provide the functionality of components included in control environment 202. These instructions executed by processors 230 may cause article computing device 110 to store and/or modify information, within storage components 236 during program execution. Processors 230 may execute instructions of components in control environment 202 to perform one or more operations in accordance with techniques of this disclosure. That is, components included in user control environment 202 may be operable by processors 230 to perform various functions described herein.

One or more communication units 232 of article computing device 110 may communicate with external devices by transmitting and/or receiving data. For example, article computing device 110 may use communication units 232 to transmit and/or receive radio signals on a radio network such as a cellular radio network. Examples of communication units 232 include a network interface card (e.g. such as an Ethernet card), an optical transceiver, a radio frequency transceiver, or any other type of device that can send and/or receive information. Other examples of communication units 232 may include Bluetooth®, 2G, 3G, 4G, Zigbee®, and Wi-Fi® radios found in mobile devices as well as Universal Serial Bus (USB) controllers and the like.

Article computing device 110 includes power source 234. In some examples, power source 234 may be a battery. Power source 234 may provide power to one or more components of article computing device 110. Examples of power source 234 may include, but are not necessarily limited to, batteries having zinc-carbon, lead-acid, nickel cadmium (NiCd), nickel metal hydride (NiMH), lithium ion (Li-ion), and/or lithium ion polymer (Li-ion polymer) chemistries. In some examples, power source 234 may have a limited capacity (e.g., 1000-2000 mAh).

One or more storage components 236 within article computing device 110 may store information for processing during operation of article computing device 110. In some examples, storage device 238 is a temporary memory, meaning that a primary purpose of storage components 236 do not include long-term storage. Storage components 236 on article computing device 110 may configured for short-term storage of information as volatile memory and therefore not retain stored contents if deactivated. Examples of volatile memories include random access memories (RAM), dynamic random access memories (DRAM), static random access memories (SRAM), and other forms of volatile memories known in the art.

Storage components 236, in some examples, also include one or more computer-readable storage media. Storage components 236 may be configured to store larger amounts of information than volatile memory. Storage components 236 may further be configured for long-term storage of information as non-volatile memory space and retain information after activate/off cycles. Examples of non-volatile memories include flash memories, or forms of electrically programmable memories (EPROM) or electrically erasable and programmable (EEPROM) memories. Storage components 236 may store program instructions and/or data associated with components included in control environment 202.

One or more input components 240 of article computing device 110 may receive input. Examples of input are tactile, audio, kinetic, and optical input, to name only a few examples. Input components 240 of article computing device 110, in one example, include an image capture device (e.g., camera), audio capture device (e.g., a microphone), presence-sensitive input device (e.g., touchpad), or any other type of device for detecting input from a human or machine.

One or more output components 242 of article computing device 110 may generate output. Examples of output are tactile, audio, and video output. Output components 242 of article computing device 110, in some examples, include a display device, audio device, vibrating device, or any other type of device for generating output (e.g., tactile, audio, and/or visual output) to a human or machine. Output components 242 may be integrated with article computing device 110 in some examples. In other examples, output components 242 may be physically external to and separate from article computing device 110, but may be operably coupled to article computing device 110 via wired or wireless communication.

Sensors 244 may include one or more movement sensors 248A, temperature sensors 248B, moisture sensors 248C, light sensors 248D, or heat flux sensors 248E. Sensors 248A-248E (collectively, sensors 248) may monitor the environment proximate computing device 110 and generate sensor information indicative of a sensed environmental characteristic and/or user physiological characteristic (e.g., air temperature, body temperature, humidity, sweat, motion, heart rate, breathing rate, etc.).

Movement sensors 248A may include one or more accelerometers, gyroscopes, etc. Movement sensors 248A generate information indicative of the movement (e.g., acceleration) of article computing device 110 in at least one plane (e.g., x, y, and/or z). Temperature sensors 248B may include one or more thermistors, resistance thermometers, thermocouples, or any other analog or digital temperature sensor. Temperature sensors 248B may generate information indicative of an internal temperature of an article of apparel (e.g., article 102 of FIG. 1), a temperature external to the article of apparel, or both. Similarly, moisture sensors 248C (e.g., a humidity sensor, such as a hygrometer, a sweat sensor, etc.) may generate information indicative of a moisture internal to article 102, external to article 102, or both. Light sensors 248D (e.g., ambient light sensors) may generate information indicative of an amount of ambient light external to article 102. Further, heat flux sensors 248E may generate information indicative of an amount of energy or heat transferred between an exterior and an interior of article 102.

Sensors 244 may continuously output sensor information or may periodically (e.g., at regular or irregular intervals) output the sensor information. For example, sensors 244 may output sensor information in response to receiving a command to output the sensor information (e.g., from sensor monitoring module 222). Outputting sensor information in response to a command for the sensor information may improve battery life relative to examples where sensors 244 continuously output sensor information.

Control environment 202 includes control logic 204 and data 206. As shown in FIG. 2, control logic 204 executes in control environment 202. Control logic 204 includes comfort prediction module 220 and sensor monitoring module 222. Data 206 includes one or more datastores. A datastore may store data in structure or unstructured form. Example datastores may be any one or more of a relational database management system, online analytical processing database, table, or any other suitable structure for storing data. As illustrated in FIG. 2, data 206 may include sensor information datastore 232A, user information datastore 232B, and article properties datastore 232C.

According to aspects of this disclosure, sensor monitoring module 222 may receive sensor information from one or more of sensors 244. In some examples, sensor monitoring module 222 continuously receives the sensor information from one or more of sensors 244. As another example, sensor monitoring module 222 may periodically (e.g., at regular or irregular intervals) receive sensor information from one or more of sensors 244. For example, sensor monitoring module 222 may receive sensor information by querying sensors 244 at regular intervals (e.g., once per minute), at irregular intervals (e.g., in response to a request for updated sensor information from comfort prediction module 120A), or a combination therein. In some examples, sensor monitoring module 222 may passively receive the sensor information (e.g., without querying sensors 244).

In some examples, sensor monitoring module 222 may store informative indicative of the sensor information to sensor information datastore 232A. The information indicative of the sensor information may include the sensor information itself, a summary of the sensor information, a subset of the sensor information, or other information that describes the sensor information. In some examples, sensor monitoring module 222 may store metadata associated with the sensor information to sensor information datastore 232A. For example, sensor monitoring module 222 may receive temperature information from temperature sensors 248B and store a timestamp indicating the time at which the temperature information was received and a value indicating the temperature at that time.

Sensor monitoring module 222 may output an indication of the sensor information to comfort prediction module 120A, user computing device 114, remote computing device 116, TPPP 112, or a combination therein. Sensor monitoring module 222 may output the indication of the sensor information automatically or in response to receiving a request for the information (e.g., from comfort prediction module 120A). In some examples, sensor 244 may output sensor information at regular or irregular intervals. In this way, sensor monitoring module 222 may manage the transfer of sensor information from sensors 244 and comfort prediction module 120A (and/or computing device 114, 116, or TPPP 112). By managing the transfer of sensor information, sensor monitoring module 222 may reduce network traffic between article computing device 110 and other computing devices, and may increase battery life of article computing device 110 (e.g., by reducing how often data is collected and/or transferred).

According to aspects of this disclosure, comfort prediction module 120A may determine (e.g., predict) whether a user of an article of apparel (e.g., article 102) is likely to be comfortable wearing one or more articles of apparel (e.g., article 102 of FIG. 1) in the future (e.g., a time in the future, later than the current time). For example, comfort prediction module 120 may determine a future time at which the individual is likely to be uncomfortable (e.g., a time at which the individual is likely to transition from comfortable to uncomfortable) while wearing article 102. As another example, comfort prediction module 120 may determine whether the individual is likely to be comfortable or uncomfortable at a particular future time (e.g., 5 pm, 3 hours from now, etc.). As used throughout this disclosure, a “time” refers to a period of time, such as a minute, several minutes, an hour, etc., rather than an instant in time.

In some examples, comfort prediction module 120 determines whether the individual is likely to be comfortable while wearing article 102 at a future time based at least in part on a thermal comfort score. Comfort prediction module 120 may determine the thermal comfort score based at least in part on sensor information received from one or more of sensors 244 and a set of one or more rules. In some examples, the set of rules is predetermined or preprogrammed (e.g., hard-coded).

In some examples, comfort prediction module 120A determines the thermal comfort score based on dynamically generated rules. For example, comfort prediction module 120A may dynamically generate the rules using machine learning (e.g., k-means clustering, SVM clustering, or other machine learning techniques) to generate at least one model that represents a plurality of environmental characteristics and/or user physiological characteristics, and user comfort levels. In some examples, comfort prediction module 120A may train the at least one model based at least in part on the sensor information. For example, sensor information datastore 232A may include historical sensor information generated by sensors 244 at a time prior to the current time

Comfort prediction module 120A may train the at least one model based on user information stored in user information datastore 232B and/or article information stored in article properties datastore 232C. In some examples, and user information datastore 232B may include historical user information received (e.g., via user input) at a time prior to the current time. In some examples, the user information includes historical user comfort information and historical user activity information. For example, the historical user comfort information may indicate whether the user was comfortable at a time prior to the current time (e.g., one week ago) or how comfortable (e.g., high, medium, low) the user was at the prior time. In some examples, historical user comfort information includes temperature control device usage information. For example, a user may manually active one or more temperature control devices 109 of article 102, as shown in FIG. 1, when the starts to feel uncomfortable, such that the temperature control device usage information may indicate whether the user was comfortable at the prior time. In some examples, temperature control device usage information includes information indicating when one or more temperature control device 109 of article 102 were activated (e.g., manually activated or deactivated by a user), a duration of use of temperature control devices 109, etc. By training the at least one model based on user information (e.g., historical user comfort information, such as temperature control device usage information), the at least one model may better predict whether and/or when a user is likely to feel comfortable.

Comfort prediction module 120A may train the at least one model based on historical user activity information. Historical user activity information may include information about the user's activity at that prior time, such as whether the user was generally physically active or generally stationary at that prior time, or a type of user physical activity (e.g., running, walking, etc.) the user was engaged in at that prior time. Article information about the article of apparel 102 worn by the user at that prior time (e.g., a type of apparel, such as a t-shirt, long-sleeved shirt, etc.), a type of material of article 102 (e.g., cotton, wool, etc.), an age of article 102, a product identifier (e.g., a universal product code (UPC)), etc. In some examples, article computing device 110 may receive the article information via user input and/or the article information may be pre-stored in article properties datastore 232C (e.g., at the time of manufacture).

Comfort prediction module 120A may apply the model to the sensor information and the historical user information stored in user information datastore 232B. For example, comfort prediction module 120A may apply the model to the current sensor information, historical sensor information, and historical user comfort information to predict whether the user of article 102 is likely to be comfortable at a time in the future while wearing article 102. In other words, the model may receive sensor information generated by sensors 244 at the current time and may output a prediction of whether the user is likely to be comfortable wearing article 102 at a particular time in the future or a prediction of when the user is not likely to be comfortable.

As one example, comfort prediction module 120A apply the model to current sensor information to identify instances where the historical sensor information is similar to the current sensor information, and determine, based on historical user comfort information for those instances, whether the user was comfortable in similar previous instances. For example, comfort prediction module 120A may receive temperature information from a first sensor (e.g., temperature sensor 248B) and moisture information (e.g., humidity information) from another sensor (e.g., moisture sensor 248C) at the current time and apply the model to the current sensor information, historical sensor information, and to the historical user information to determine the thermal comfort score. For example, the at least one model may indicate the user typically remains comfortable for long periods of time when the temperature is 75° F. and the dew point of 50° F. In such examples, comfort prediction module 120A may assign a relatively high thermal comfort score (e.g., 90 of 100, which may indicate a user of article 102 is likely comfortable) for a given temperature (e.g., 75° F.) and humidity (e.g., a dew point of 50° F.). As another example, comfort prediction module 120A may apply the at least one model to different sensor information (e.g., indicating a temperature of 55° F. and a dew point of 50° F.) and may assign a different (e.g., lower) thermal comfort score (e.g., 40 out of 100).

Comfort prediction modules 120 may determine the thermal comfort score based on a comparison of the sensor information to a threshold value associated with the sensor information. For example, the sensor information may include temperature information when temperature sensor 248B detects the temperature external to article 102 (e.g., the ambient air temperature) or the temperature internal to article 102 (e.g., the air temp between a wearer's body and the inside surface of article 102). In such examples, comfort prediction module 120A may determine the thermal comfort score based on the received temperature information and a temperature threshold. For example, comfort prediction module 120A may assign a relatively high thermal comfort score (e.g., 90 out of 100, which may indicate the user of article 102 is likely comfortable) when the temperature equals a threshold temperature (e.g., 70° Fahrenheit) or when the temperature is within a range of temperatures defined by an upper temperature threshold (e.g., 70° F.) and a lower temperature threshold (e.g., 60° F.). Comfort prediction module 120A may assign a lower thermal comfort score as the temperature increases above a temperature threshold (e.g., which may indicate the user is likely uncomfortably hot) or as the temperature decreases below a temperature threshold (e.g., which may indicate the user is likely uncomfortably cold).

In some examples, comfort prediction module 120A dynamically determines the one or more temperature thresholds. For example, comfort prediction module 120A may determine the temperature threshold based on movement information received from a movement sensor 248A. For example, comfort prediction module 120A may determine that the user is engaged in a physical activity (e.g., an athletic activity or exercise, such as running, walking, weight lifting, yoga, etc.) based on the movement information and may assign a threshold temperature in response to determining that the user is engaged in a physical activity (e.g., running). In some examples, comfort prediction module 120A may set the threshold temperature to a particular temperature (e.g., 60° F.) or set a threshold temperature range (e.g., between 55° F. and 65° F.) in response to determining that the user is engaged in a physical activity. For example, the historical user information may include an indication of whether the user of the article of apparel was engaged in a physical activity and whether the individual was comfortable at that time. In such examples, comfort prediction module 120A may apply the model to the sensor information generated at the current time and to the historical user information to generate the thermal comfort score for the current time.

In some examples, comfort prediction module 120A may determine the thermal comfort score based at least in part on information about a particular article of apparel. For example, comfort prediction module 120A may determine the thermal comfort score based on a type of the article 102 worn by the individual, an age of the article 102, a type of material of article 102, etc. For instance, comfort prediction module 120A may receive an indication of the particular article 102 worn by the user (e.g., via a user input at user computing device 114) and may query article properties data store 232C for information about article 102 (e.g., year of purchase or manufacture, type of material, etc.). For example, comfort prediction module 120 may assign a lower thermal comfort score as article 102 ages (e.g., because the insulation properties of article 102 may degrade over time). Similarly, in some examples, different types of material may have different thermal properties (e.g., different R-values) such that user comfort module 120A may determine the thermal comfort score based on the type of material of article 102. For example, comfort prediction module 120A may assign a first thermal comfort score when the type of material of article 102 is a first type of material (e.g., cotton) and a different (e.g., higher) thermal comfort score when the type of material is a different type of material (e.g., wool).

Responsive to determining the thermal comfort score, comfort prediction module 120A may determine whether the thermal comfort score satisfies (e.g., is greater than or equal to) a threshold comfort score. In some examples, comfort prediction module 120A determines or predicts that the user is likely to be comfortable wearing article 102 at a future time in response to determining that the thermal comfort score satisfies (e.g., is greater than or equal) the threshold comfort score. Similarly, comfort prediction module 120A may determine or predict that the user is not likely to be comfortable at the future time in response to determining that the thermal comfort score does not satisfy (e.g., is less than) the threshold comfort score.

Responsive to determining or predicting that the user is not likely to be comfortable at a future time while wearing article 102, comfort prediction module 120A may cause a computing device to perform one or more operations. In some examples, the one or more operations include outputting a notification indicative of the prediction that the individual is not likely to be comfortable at some time in the future. As one example, comfort prediction module 120A may output the notification to an output device of article 102 (e.g., a graphical, audio, and/or haptic user interface device) or to another computing device (e.g., user computing device 114 and/or remote computing device 116).

In some examples, the one or more operations include adjusting operation of a temperature control device of the article of apparel. For example, comfort prediction module 120A may automatically activate a heating device. For example, when the heating device includes a resistive wire that generates heat when passing a current, article computing device 110 may output a command that causes a current through the insulated wire to cause the heating device to output heat.

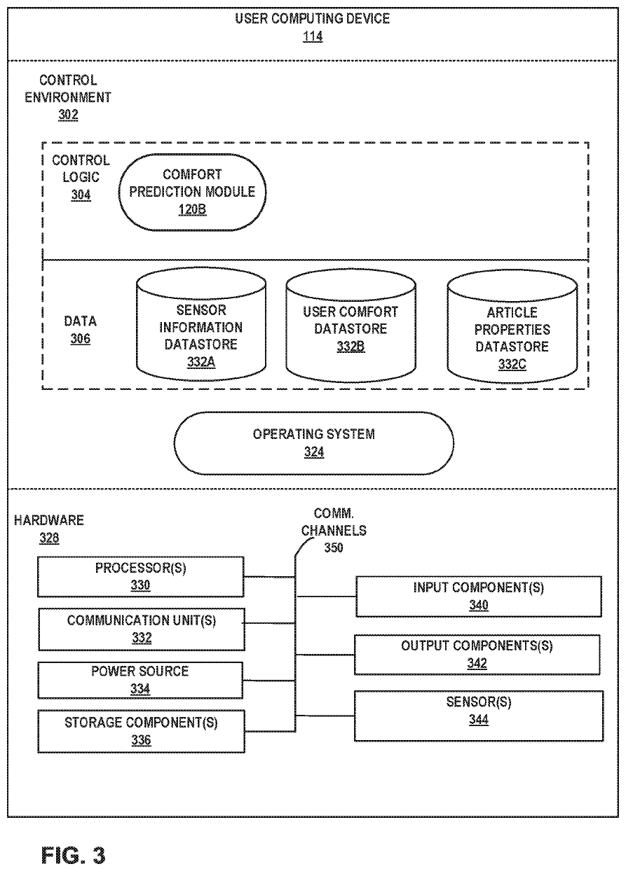

FIG. 3 is a block diagram illustrating an example computing device that is configured to predict whether a user of an article of apparel will be comfortable at a future time, in accordance with various techniques of this disclosure. FIG. 3 illustrates only one particular example of user computing device 114. Many other examples of user computing device 114 may be used in other instances and may include a subset of the components illustrated in FIG. 3 and/or may include additional components not shown in FIG. 3.

User computing device 114 may be logically divided into control environment 302 and hardware 328. Hardware 328 may include one or more hardware components that provide an operating environment for components executing in control environment 302. Control environment 302 may include operating system 324, which or may not operate with higher privileges than other components executing in control environment 302.

As shown in FIG. 3, hardware 328 includes one or more processors 330, communication units 332, power source 334, storage components 336, input components 340, output components 342, and sensors 344. Processors 330, communication units 332, power source 334, storage components 336, input components 332, output components 342, and sensors 344 may each be interconnected by one or more communication channels 250. Communication channels 250 may interconnect each of the components 330, 332, 334, 336, 340, 342, and 344 for inter-component communications (physically, communicatively, and/or operatively). In some examples, communication channels 250 may include a hardware bus, a network connection, one or more inter-process communication data structures, or any other components for communicating data between hardware and/or software. Processors 330, communication units 332, power source 334, storage components 336, input components 340, output components 342, and sensors 344 may be similar to and include functionality similar to processors 230, communication units 232, power source 234, storage components 236, input components 232, output components 242, and sensors 244 of FIG. 2. Thus, a description of processors 330, communication units 332, power source 334, storage components 336, input components 340, output components 342, and sensors 344 is omitted for brevity.

Control environment includes control logic 304 and data 306. Data 306 may include sensor information datastore 332A, user comfort information datastore 332B, and article properties datastore 332C, which may include information similar to sensor information datastore 232A, user comfort information datastore 232B, and article properties datastore 232C, respectively, as described with reference to FIG. 2. In some examples, control logic 306 includes comfort prediction module 120B. Comfort prediction module 120B may include the functionality of comfort prediction module 120A described in FIGS. 1 and 2.

According to aspects of this disclosure, comfort prediction module 120B may output, for display, a graphical user interface including graphical elements (e.g., a text box, radio buttons, etc.) requesting information from a user of user computing device 114. In some examples, the graphical user interface may include a graphical element requesting user comfort information indicative of whether the user is comfortable at a first time. For example, the graphical user interface may include a plurality of radio buttons labeled “very comfortable”, “somewhat comfortable”, or “not comfortable”. In some examples, the graphical user interface includes a graphical element requesting user activity information indicative of whether the user is physically active or what type of activity the user is engaged in at the first time. For example, the graphical user interface may include radio buttons labeled “running”, “walking”, “skiing”, “other exercise”, or “relaxing”. In some examples, the graphical user interface includes one or more graphical elements requesting article information about one or more articles of apparel worn by the user. For example, the graphical user interface may include menus enabling the user to select a type of material, age, manufacture, type of article (e.g., shirt, pants, long-sleeved shirt, short-sleeved shirt, etc.). Comfort prediction module 120B may output the GUI information to an output component 342 (e.g., a display device), such that output component 342 displays the GUI information as a graphical user interface.

User computing device 114 may receive a user input indicative of user information and/or article information at the first time. For example, the user of computing device 114 may input user comfort information (e.g., by selecting a graphical element of the GUI) indicating whether the user is comfortable at the first time (or how comfortable the user is at the first time), user activity information indicating whether the user is physically active or what type of activity the user is engaged in at the first time, article information indicating information about one or more articles of apparel worn by the user at the first time, or any combination therein. Comfort prediction module 120B may receive the user information and/or article information and store the information in user comfort datastore 332B or article properties datastore 332C, respectively.

In some examples, at a second time that is later than the first time (e.g., several hours after the first time), comfort prediction module 120B may predict whether the user is likely to be comfortable wearing one or more articles of apparel (e.g., article 102 of FIG. 1) at a future (e.g., third) time. Comfort prediction module 120B may determine whether the user is likely to be comfortable wearing the article 102 at the future time in a manner similar to comfort prediction module 120A described with reference to FIGS. 1 and 2. For example, comfort prediction module 120B may receive sensor information generated one or more sensors (e.g., sensors 244 of article computing device 110 and/or sensors 344 of user computing device 114) at the second time. In some examples, the sensor information generated at the second time may be referred to as the current sensor information. Comfort prediction module 120B may determine, based at least in part on the sensor information generated at the second time, whether the user is likely to be comfortable at a future, third time. In some examples, comfort prediction module 120B may apply a set of rules to the current sensor information to determine a thermal comfort factor indicative of a probability the user will be comfortable at the future time. For example, comfort prediction module 120B may assign the thermal comfort score by applying one or more models generated by machine learning to the current sensor information. In some examples, comfort prediction module 120B may apply the one or more models to the current sensor information and to historical sensor information stored at datastore 332A (e.g., sensor information generated and received by one or more sensors at a previous time(s)), historical user information stored at datastore 332B (e.g., user comfort information and/or user activity information received at a previous time(s)), article information stored at datastore 332C, or a combination therein.

In some examples, comfort prediction module 120B may determine whether the user is likely to be comfortable at a future time based on information associated with a plurality of articles of apparel. For example, comfort prediction module 120B may receive an indication of user input (e.g., via a graphical user interface) indicating the user is wearing a set of articles of apparel (e.g., a sweatshirt, a jacket, snowpants, and boots), may receive information about a plurality of articles in the set of articles (e.g., the sweatshirt and the jacket), and may determine the thermal comfort score based on the information associated with the plurality of articles. For instance, comfort prediction module 120B may determine that the user is likely to stay warm for a longer period of time when wearing a jacket and sweatshirt.

Responsive to determining, at the second time, the thermal comfort score associated with a future, third time, comfort prediction module 120B may determine whether the thermal comfort score satisfies (e.g., is greater than) a threshold comfort score. In some examples, comfort prediction module 120B determines the user is not likely to be comfortable at the future time while wearing article 102 in response to determining that the thermal comfort score does not satisfy (e.g., is less than) the thermal comfort score. In some examples, comfort prediction module 120B determines that the user is likely to be comfortable at the future time while wearing article 102 in response to determining that the thermal score satisfies (e.g., is great than) the threshold comfort score.

In some examples, comfort prediction module 120B performs at least one operation in response to determining that the user is not likely to be comfortable at the future time. For example, comfort prediction module 120B may output a notification (e.g., as a text message, email, etc.) indicating the user is not likely to be comfortable at the future time to another computing device (e.g., remote computing device 116 of FIG. 1). As another example, comfort prediction module 120B may output a notification (e.g., visual, audible, etc.) via an output component (e.g., display device, audio device) of user computing device 114. In some examples, comfort prediction module 120B outputs a notification or command to article computing device 110. For example, comfort prediction module 120B may output, to article 102 of FIG. 1, a command to adjust operation of a temperature control device 109 of article 102. For instance, the command may include a command to activate a heating device or a cooling device, such that article 102 may better regulate the temperature and comfort level of the user.

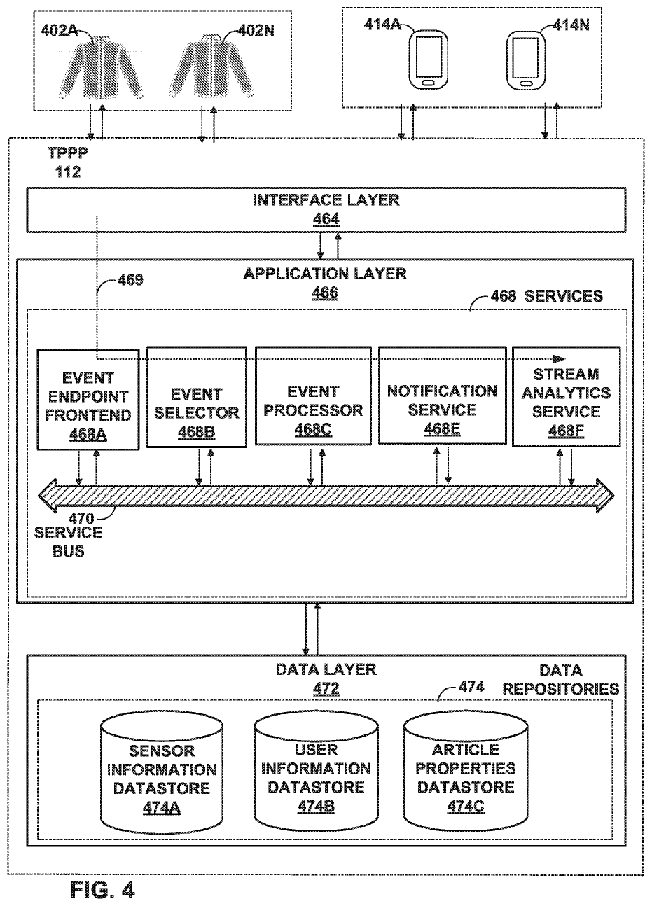

FIG. 4 is a block diagram providing an operating perspective of an example TPPP that is configured to predict whether a user of an article of apparel will be comfortable at a future time, in accordance with various techniques of this disclosure. TPPP 112 may be configured to communicate with a plurality of articles of apparel 402A-402N (collectively, “articles 402”), a plurality of computing devices 414A-414N (collectively, “computing devices 414”), or a combination therein. Articles 402 may be examples of article 102 of FIG. 1. Computing devices 414 may be examples of user computing device 114 and/or remote computing device 116 of FIG. 1. In the example of FIG. 4, the components of TPPP 112 are arranged according to multiple logical layers that implement the techniques of the disclosure. Each layer may be implemented by one or more modules comprised of hardware, hardware and software, hardware and firmware, or a combination therein.

In FIG. 4, articles 402 and computing devices 414 may communicate with TPPP 112 via interface layer 464. Computing devices 414 typically execute client software applications, such as desktop applications, mobile application, and web applications. Examples of computing devices 414 may include portable or mobile computing device (e.g., smartphone, wearable computing device, tablet), laptop computers, desktop computers, smart television platforms, and servers, to name only a few examples.

As further described in this disclosure, articles 402 communicate with TPPP 112 (directly or via computing devices 414) to provide streams of information acquired from embedded sensors and other monitoring circuitry and may receive alerts, configuration information, and other communications from TPPP 112. Client applications executing on computing devices 414 may communicate with TPPP 112 to send and receive information that is retrieved, stored, generated, and/or otherwise processed by services 468. For instance, the client applications may request and edit information (e.g., sensor information, user information, and/or article information) stored at and/or managed by TPPP 112. In some examples, client applications of computing devices 414 may request a predicted thermal comfort score. In some examples, the client applications may output (e.g., for display) information received from TPPP 112 to visualize such information for users of computing devices 414. As further illustrated and described in below, TPPP 112 may provide information to the client applications, which the client applications output in user interfaces (e.g., audio, graphical, etc.).

Clients applications executing on computing devices 414 may be implemented for different platforms but include similar or the same functionality. For instance, a client application may be an application compiled to run on an operating system (e.g., desktop or mobile operating system). As another example, a client application may be a web application such as a web browser that displays web pages received from TPPP 112 (e.g., TPPP 112 may, in some examples, represent a “cloud” computing system). In the example of a web application, TPPP 112 may receive requests from the web application (e.g., the web browser), process the requests, and send one or more responses back to the web application. In this way, the collection of web pages, the client-side processing web application, and the server-side processing performed by TPPP 112 collectively provides the functionality to perform techniques of this disclosure. In this way, client applications use various services of TPPP 112 in accordance with techniques of this disclosure, and the applications may operate within various different computing environment (e.g., embedded circuitry or processor of an article of apparel, a desktop operating system, mobile operating system, or web browser, to name only a few examples).

As shown in FIG. 4, TPPP 112 includes an interface layer 464 that represents a set of application programming interfaces (API) or protocol interface presented and supported by TPPP 112. Interface layer 464 initially receives messages from any of articles 402 and/or computing devices 414 for further processing at TPPP 112. Interface layer 464 may provide one or more interfaces that are available to client applications executing on computing devices 414. In some examples, the interfaces may be application programming interfaces (APIs) that are accessible over a network. Interface layer 464 may be implemented with one or more web servers. The one or more web servers may receive incoming requests, process and/or forward information from the requests to services 468, and provide one or more responses, based on information received from services 468, to the client application that initially sent the request. In some examples, the one or more web servers that implement interface layer 464 may include a runtime environment to deploy program logic that provides the one or more interfaces. As further described below, each service may provide a group of one or more interfaces that are accessible via interface layer 464.

In some examples, interface layer 464 may provide Representational State Transfer (RESTful) interfaces that use HTTP methods to interact with services and manipulate resources of TPPP 112. In such examples, services 468 may generate JavaScript Object Notation (JSON) messages that interface layer 464 sends back to the client application that submitted the initial request. In some examples, interface layer 464 provides web services using Simple Object Access Protocol (SOAP) to process requests from client applications. In still other examples, interface layer 464 may use Remote Procedure Calls (RPC) to process requests from computing devices 414. Upon receiving a request from a client application to use one or more services 468, interface layer 464 sends the information to application layer 466, which includes services 468.

As shown in FIG. 4, TPPP 112 also includes an application layer 466 that represents a collection of services 468 for implementing much of the underlying operations of TPPP 112. Application layer 466 receives information included in requests received from the client applications and further processes the information according to one or more of services 468 invoked by the requests. Application layer 466 may be implemented as one or more discrete software services executing on one or more application servers, e.g., physical or virtual machines. That is, the application servers provide runtime environments for execution of services 468. In some examples, the functionality interface layer 464 as described above and the functionality of application layer 466 may be implemented at the same server.

Application layer 466 may include one or more separate software services 468, e.g., processes that communicate, e.g., via a logical service bus 470 as one example. Service bus 470 generally represents a logical interconnections or set of interfaces that allows different services to send messages to other services, such as by a publish/subscription communication model. For instance, each of services 468 may subscribe to specific types of messages based on criteria set for the respective service. When a service publishes a message of a particular type on service bus 470, other services that subscribe to messages of that type will receive the message. In this way, each of services 468 may communicate information to one another. As another example, services 468 may communicate in point-to-point fashion using sockets or other communication mechanism. Before describing the functionality of each of services 468, the layers are briefly described herein.

Data layer 472 of TPPP 112 represents a data repository that provides persistence for information in TPPP 112 using one or more data repositories (also referred to as datastores) 474. A data repository, generally, may be any data structure or software that stores and/or manages data. Examples of data repositories include but are not limited to relational databases, multi-dimensional databases, maps, and hash tables, to name only a few examples. Data layer 472 may be implemented using Relational Database Management System (RDBMS) software to manage information in data repositories 474. The RDBMS software may manage one or more data repositories 474, which may be accessed using Structured Query Language (SQL). Information in the one or more databases may be stored, retrieved, and modified using the RDBMS software. In some examples, data layer 472 may be implemented using an Object Database Management System (ODBMS), Online Analytical Processing (OLAP) database or other suitable data management system.

As shown in FIG. 4, each of services 468A-468F (“services 468”) is implemented in a modular form within TPPP 112. Although shown as separate modules for each service, in some examples the functionality of two or more services may be combined into a single module or component. Each of services 468 may be implemented in hardware, hardware and software, hardware and firmware, or a combination therein. Moreover, services 468 may be implemented as standalone devices, separate virtual machines or containers, processes, threads or software instructions generally for execution on one or more physical processors.

In some examples, one or more of services 468 may each provide one or more interfaces that are exposed through interface layer 464. Accordingly, client applications of computing devices 414 may call one or more interfaces of one or more of services 468 to perform techniques of this disclosure.

In accordance with techniques of the disclosure, services 468 may include an event processing platform including an event endpoint frontend 468A, event selector 468B, event processor 468C, notification service 468E, and stream analytics service 468F. Event endpoint frontend 468A operates as a front-end interface for receiving and sending communications to articles 402 and computing devices 414. In some instances, event endpoint frontend 468A may be implemented as a plurality of tasks or jobs spawned to receive individual inbound communications of event streams 469 (e.g., streams of information). When receiving event streams 469, for example, event endpoint frontend 468A may spawn tasks to quickly enqueue an inbound communication, referred to as an event, and close the communication session, thereby providing high-speed processing and scalability. Each incoming communication may, for example, carry sensor information generated by sensors embedded within various articles 402 or and/or by sensors of computing devices 414, user information (e.g., user comfort information and/or user activity information), article information, or a combination therein. Communications exchanged between the event endpoint frontend 468A and the articles 402 and/or computing devices 414 may be real-time or pseudo real-time depending on communication delays and continuity.

Event selector 468B operates on the stream of events 469 received from articles 402 and/or computing devices 414 via frontend 468A and determines, based on rules or classifications, priorities associated with the incoming events. Based on the priorities, event selector 468B enqueues the events for subsequent processing by event processor 468C.

In general, event processor 468C operates on the incoming streams of events to update event data 474A within data repositories 474. In general, event data 474A may include all or a subset of sensor information obtained from articles 402. For example, in some instances, event data 474A may include entire streams of samples of data obtained from sensors of articles 402 (e.g., from temperature sensors, light sensors, etc., as described with reference to sensors 108 of FIG. 1). In other instances, event data 474A may include a subset of such data, e.g., associated with a particular time period or activity articles 402.

Event processor 468C may create, read, update, and delete information stored in data repositories 474. Data repositories 474 may store information in structured or unstructured form. Example data repositories may be any one or more of a relational database management system, online analytical processing database, table, or any other suitable structure for storing data. For example, sensor information datastore 474A may include sensor information generated by sensors of a plurality of articles 402. User information datastore 474B may include user information associated with the sensor information. Similarly, article properties datastore 474C may include article information associated with the sensor information and user information. For example, article properties datastore 474C may include an article identifier (e.g., UPC code), article type (e.g., t-shirt, long-sleeved shirt, jacket, etc.), type of material for the article (e.g., wool, cotton, etc.), age of the article, etc.

Event processor 468C may associate (e.g., link) sensor information, user information, and/or article information. In other words, datastores 474 may associate (e.g., via relational databases) sensor information generated at a previous time by sensors of a particle article of articles 402, corresponding user information associated with a user of the particular article at the previous time, and article information associated with the particular article. As one example, datastores 474 may include acceleration information generated by sensors of article 402A on June 1, corresponding user information associated with a particular user who wore the article 402A on June 1, and article information for article 402A. Similarly, datastores 474 may include acceleration information generated by sensors of article 402B on June 5, corresponding user information with another user who wore article 402B on June 5, and article information for article 402B, and so on.

Event selector 468B directs the incoming stream of events to stream analytics service 468F, which is configured to perform in depth processing of the incoming stream of events to perform real-time analytics. Stream analytics service 468F may, for example, be configured to process and compare multiple event stream 469 in real-time as the event streams are received. Analytics service 468F may receive and processes many inbound streams of events (e.g., potentially hundreds or thousands of streams of events) from enabled articles 402 and/or computing devices 414 to predict whether a particular individual is likely to be comfortable at a future time while wearing one or more of articles 402.

According to aspects of this disclosure, TPPP 112 may determine whether a user of one or more articles 402 is likely to be comfortable at a future time while wearing one or more articles 402. For example, stream analytics service 468F may determine a thermal comfort score associated with a user of one or more articles 402 based at least in part on the sensor information in event streams 469. Responsive to determining the thermal comfort score, stream analytics service 468F may determine whether the user of one or more articles 402 is likely to be comfortable at some time in the future.

Stream analytics service 468F determines the thermal comfort score based on a set of rules. In some examples, the set of rules are preprogrammed. As another example, stream analytics service 468F may dynamically generate the set of rules using machine learning to generate at least one model that represents a plurality of environmental characteristics and/or user physiological characteristics, and user comfort levels. Example machine learning techniques that may be employed to generate the at least one model can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbour (kNN), Learning Vector Quantization (LUQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, stream analytics service 468F trains the at least one model based at least in part on sensor information stored in sensor information datastore 474A and user information stored in user information datastore 474B. For example, stream analytics service 468F may apply the model and determine based at least in part on the sensor information and user information that users of various articles 402 tend to stay comfortable longer, or are more comfortable, when the ambient air temperature is within a threshold range of temperatures bounded by an upper temperature threshold and a lower temperature threshold.

Stream analytics service 468F may train the at least one model based at least in part on article information stored in article properties datastore 474C. For example, stream analytics service 468F may apply the model and determine that users tend to be more comfortable or stay comfortable longer in certain types of material. As another example, stream analytics service 468F may apply the model and determine that users become uncomfortable faster when the articles 402 reach a threshold age.

Stream analytics service 468F may determine (e.g., in real-time or approximately real-time), based on the one or more models, whether a user of one or more articles 402 is likely to be comfortable wearing one or more articles 402 at a time in the future. Stream analytics service 468F may apply the model to sensor information, user information, article information, or any combination therein, to determine the thermal comfort score.

For example, stream analytics service 468F determines a thermal comfort score by applying the model to sensor information generated by sensors of one or more articles 402 at the current time, sensor information stored in sensor information datastore 474A (e.g., generated and stored at previous times), and user information stored in user information datastore 474B (e.g., stored at previous times). In other words, the one or more models may receive sensor information generated by one or more sensors of a given article of apparel 402 at the current time and may output a predicted thermal comfort score indicative of a probability that the user is likely to be comfortable at a future time. For example, when the sensor information includes humidity information, stream analytics service 468F apply the model and determine that humidity has relatively small effects on user comfort at relatively low temperatures (e.g., below a threshold temperature, such as 50° F.) and affects user comfort more as temperatures increase (e.g., increase above the threshold temperature). Thus, stream analytics service 468F may receive sensor information generated by a particular article of articles 402 at the current time and determine, based on applying the model to the sensor information for the current time, the user information (e.g., historical user comfort information) stored in datastore 474B, and the sensor information stored in datastore 474A, a thermal comfort score associated with the user that is wearing the particular article of apparel 402 at the current time.

In some examples, stream analytics service 468F determines the thermal comfort score based at least in part on article information stored in article properties datastore 474C. For example, TPPP 112 may receive sensor information generated by one or more sensors of a particular article 402 at the current time and article information about the particular article (e.g., an age of the article, type of material of the article, etc.). Stream analytics service 468F may apply the one or more models to the sensor information generated at the current time, the article information associated with the particular article, sensor information stored in datastore 474A, and article information stored in datastore 474C and may output a predicted thermal comfort score.

In some examples, when the current sensor information is generated by a particular article 402A, stream analytics service 468F may generate the thermal comfort score by applying the model to information (sensor information, user information, and/or article information) associated with only the user of article 402A. In other words, the thermal comfort score may be based on information that is not associated with users of other articles 402. In some examples, stream analytics service 468F generates the thermal comfort score by applying the model to information associated with a plurality of users of a plurality of respective articles 402. In other words, the thermal comfort score may be based on information (e.g., sensor information, user information, and/or article information) associated with a group of users of articles 402.

Responsive to determining the thermal comfort score, stream analytics service 468F may determine whether the thermal comfort score satisfies (e.g., is greater than or equal to) a threshold comfort score. Stream analytics service 468F may determine the threshold comfort score by querying a memory device (e.g., the threshold comfort score may be hard-coded). In some examples, stream analytics service 468F dynamically determines the threshold comfort score in a manner similar to the technique described with reference to comfort prediction module 120A of FIGS. 1 and 2.

Stream analytics service 468F may determine that the user is likely to be comfortable at a future time while wearing one or more articles 102 in response to determining that the thermal comfort score satisfies (e.g., is greater than or equal) the threshold comfort score. Similarly, stream analytics service 468F may determine that the user is not likely to be comfortable at the future time while wearing one or more articles 102 in response to determining that the thermal comfort score does not satisfy (e.g., is less than) the threshold comfort score.

Responsive to determining or predicting that the user is not likely to be comfortable at a future time, notification service 468E may cause a computing device to perform one or more operations. For example, notification service 468E may output a notification indicative of the prediction that the individual is not likely to be comfortable at some time in the future. As one example, notification service 468E may output the notification to a computing device of article 402A and/or one or more computing devices 414. In this way, article 402A and/or one or more computing devices 414 may receive the notification and output an alert indicating that the user is not likely to be comfortable at the future time. For example, one of computing devices 414 may output a GUI that includes an alert indicating the user is not likely to be comfortable by a future time. For example, notification service 468E may output the notification for display as a graphical user interface at a display device of one or more computing devices 414. For instance, the notification may cause a computing device of computing devices 414 to display a graphical user interface that includes a dashboard, an alert, a report, or the like. Such information may provide various insights regarding comfort levels for one or more individuals wearing one or more articles 402.

In some examples, notification service 468E may output a command to one or temperature control devices of article 402A to adjust operation of the temperature control device in response to determining that the individual is predicted to be uncomfortable. In some examples, the command may include a command to automatically activate a heating device. For example, when the heating device includes a resistive wire that generates heat when passing a current, notification service 468E may command to article 402 (e.g., directly or via one of computing device s414) to output a current through the insulated wire to cause the heating device to output heat.

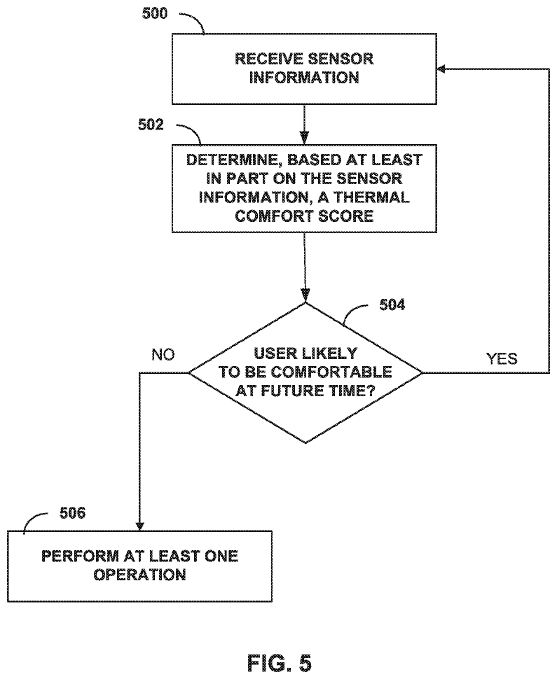

FIG. 5 is a flow chart illustrating example operations performed by one or more computing devices that are configured to predict whether a user of an article of apparel will be comfortable at a future time. While the steps shown in FIG. 5 are exemplary steps associated with the present disclosure, variations on the order of the steps, and additional steps, will be apparent to one of skill in the art upon reading the present disclosure. For ease of illustration only, the method of FIG. 5 is described with reference to system 100 of FIG. 1, however other example systems may perform the method.

A method includes receiving, by at least one processor, sensor information generated by at least one sensor 108 embedded in article of apparel 102 (500). For example, at least one sensor 108 of article 102 may generate sensor information and output the sensor information to article computing device 110, such that article computing device 110 may receive the sensor information. As another example, user computing device 114, remote computing device 116, TPPP 112, or any combination therein may receive the sensor information (e.g., directly or indirectly) from article computing device 110.

The method includes determining, by the at least one processor, based at least in part on the sensor information, a thermal comfort score indicative of a probability that a user of the article of apparel will be comfortable at a future time (502). For example, article computing device 110 may determine the thermal comfort score. As another example, user computing device 114, remote computing device 116, TPPP 112, or any combination therein may determine the thermal comfort score in response to receiving the sensor information. In some examples, the at least one processor determines the thermal comfort score by applying a set of rules to the sensor information. The set of rules may be preprogrammed or dynamically generated by the at least one processor using machine learning. In some examples, the at least one processor determines the thermal comfort score based at least in part on user information (e.g., historical user comfort information, user activity information, etc.) and/or article information (e.g., age, type of material, etc.).

The at least one processor may predict, based on the thermal comfort score, whether the user of the article of apparel is likely to be comfortable at the future time (504). For example, the at least one processor may determine whether the user is likely to be comfortable at some in in the future or determine a time at which the user is not likely to be comfortable (e.g., an approximate time at which the user is likely to transition from comfortable to uncomfortable). The at least one processor of article computing device 110, user computing device 114, remote computing device 116, TPPP 112, or any combination therein, may compare the thermal comfort score to a threshold comfort score. The at least one processor may predict that the user is likely to be comfortable at the future time when the thermal comfort score satisfies (e.g., is greater than or equal to) the threshold comfort score. The at least one processor may predict that the user is not likely to be comfortable at the future time when the thermal comfort score does not satisfy the threshold comfort score.

The method includes performing at least one operation (506) in response to determining that the user of the article of apparel is not likely to be comfortable at the future time (“NO” branch of 504). In some examples, performing at least one operation includes outputting a notification indicating the user is not likely to be comfortable at a future time. For example, article computing device 110 may output the notification to another computing device (e.g., user computing device 114). As another example, user computing device 114 may output the notification via an output device of computing device 114 (e.g., as a graphical user interface).

Responsive to determining that the user is likely to be comfortable (“YES” branch of 504), the method includes returning to (500). In other words, the at least one processor may continue to receive sensor information and update the thermal comfort score based at least in part on the newly received sensor information.

While article computing device 110, user computing device 114, and TPPP 112 are described as determining whether a user is likely to be comfortable at a future time, in some examples, computing device 110, user computing device 114, and/or TPPP 112 may recommend an article of apparel based on similar information and rules as described above. For example, article computing device 110 may determine, from a set of articles of apparel, one or more articles of apparel that are likely to keep the user comfortable for a given set of conditions. For instance, article computing device 110 may receive a request for assistance identifying one or more articles of apparel that are likely to keep the user comfortable during a future period of time. Article computing device 110 may receive the request, which may include information associated with the future period of time, such as information indicating the period of time (e.g., indicating a date, amount of time, window of time such as 12 pm-5 pm, etc.), information indicative of a physical activity to be performed by the user during that future period of time, weather information for the forecasted weather during that future period of time, and the like. Article computing device 110 may determine based on the received information, one or more articles of apparel that are likely to keep the user comfortable during that period of time. Article computing device 110 may apply the rules described above to any of the types of information (e.g., historical sensor information, historical user information, article information, etc.) to identify one or more articles. For example, article computing device 110 may apply the one or more models to the historical user comfort information, historical sensor information, article information, and the information associated with the future period of time. The model may output one or more articles of apparel that are likely to enable the user to remain comfortable during the future period of time. For example, article computing device 110 may apply the model and output information identifying a set of articles that, separately or in combination, may enable the user to remain comfortable during the future period of time.

Although the methods and systems of the present disclosure have been described with reference to specific exemplary embodiments, those of ordinary skill in the art will readily appreciate that changes and modifications may be made thereto without departing from the spirit and scope of the present disclosure.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

1. A method comprising:
receiving, by at least one processor, sensor information generated by a sensor embedded in an article of apparel;
determining, by the at least one processor, based at least in part on the sensor information, a thermal comfort score indicative of a probability that a user of the article of apparel will be comfortable at a future time;
determining, by the at least one processor, based on the thermal comfort score, whether the user of the article of apparel is likely to be comfortable at the future time; and
responsive to determining that the user of the article of apparel is not likely to be comfortable at the future time, performing an operation.

2. The method of claim 1, wherein performing the operation comprises:
outputting, by the at least one processor, a notification indicating that the individual is not likely to be comfortable at the future time.

3. The method of claim 1, wherein performing the operation comprises:
adjusting a component of the article of apparel.

4. The method of claim 3, wherein adjusting the component comprises automatically activating a heating device or a cooling device of the article of apparel.

5. The method of claim 1, wherein determining the thermal comfort score is further based on a set of one or more predetermined rules.

6. The method of claim 1, further comprising:
receiving user comfort information indicative of a user comfort of a user at a first time;
wherein determining the thermal comfort score comprises:
applying, by the at least one processor and at a second time later than the first time, a model to at least the sensor information and the user comfort information to determine the thermal comfort score.

7. The method of claim 1, wherein determining the thermal comfort score comprises:
determining, by the at least one processor, based on applying a model to the sensor information and user comfort information received from a plurality of users, the thermal comfort score,
wherein the user comfort information received from each user of the plurality of users is indicative of user comfort for the respective user at a respective time that is earlier than a current time.

8. The method of claim 1, wherein determining the thermal comfort score is further based on a type of material of the article of apparel.

9. The method of claim 1, wherein determining the thermal comfort score is further based on an age of the article of apparel.

10. The method of claim 1, wherein the sensor is a first sensor, wherein the method comprises:
receiving, by the at least one processor, sensor information generated by a second sensor embedded in an article of apparel,
wherein determining the thermal comfort score is based on the information generated by the first sensor and the second sensor.

11. The method of claim 10, wherein the first sensor is disposed on a first surface of the article of apparel and the second sensor is disposed on a second, different surface of the article of apparel or between an inner surface and an exterior surface of the article of apparel.

12. The method of claim 1, wherein the sensor comprises one or more of:
a temperature sensor,
a humidity sensor,
a movement sensor,
a heat flux sensor,
a heart rate sensor, or
an ambient light sensor.

13. A system comprising:
an article of apparel comprising a sensor;
at least one processor; and
a memory comprising instructions that, when executed by the at least one processor, causes the at least one processor to:
receive sensor information generated by the sensor;
determine, based at least in part on the sensor information, a thermal comfort score indicative of a probability that a user of the article of apparel will be comfortable at a future time;
determine, based on the thermal comfort score, whether the user of the article of apparel is likely to be comfortable at the future time; and
responsive to determining that the user of the article of apparel is not likely to be comfortable at the future time, perform an operation.

14. The system of claim 13, wherein execution of the instructions causes the at least one processor to perform the operation by causing the at least one processor to:
output a notification indicating that the user of the article is not likely to be comfortable at the future time.

15. The system of claim 13, wherein execution of the instructions causes the at least one processor to perform the operation by causing the at least one processor to:
adjust a component of the article of apparel.

16. The system of claim 13, wherein execution of the instructions further causes the at least one processor to receive user comfort information indicative of a user comfort at a first time;
wherein execution of the instructions causes the at least one processor to determine the thermal comfort score by at least causing the at least one processor to apply, at a second time later than the first time, a model to at least the sensor information to determine the thermal comfort score.

17. The system of claim 13, wherein execution of the instructions causes the at least one processor to determine the thermal comfort score further based on applying a model to the sensor information and user comfort information received from a plurality of users,
wherein the user comfort information received from each user of the plurality of users is indicative of user comfort of the respective user at a respective time that is earlier than a current time.

18. The system of claim 13, wherein the sensor comprises one or more of:
a temperature sensor,
a moisture sensor,
a movement sensor,
a heat flux sensor,
a heart rate sensor, or
an ambient light sensor.

19. The system of claim 13, wherein the article of apparel includes the memory and the at least one processor.

20. A computer-readable storage medium, comprising instructions that when executed by at least one processor of a computing device, causes the at least one processor to perform the method of claim 1.
